(19) United States
(12) Patent Application Publication
(10) Pub. No.: US 2024/0285853 A1
(43) Pub. Date: Aug. 29, 2024

(54) METHODS AND DEVICES FOR DETECTING INTRAVENOUS INFUSION INFILTRATION

(71) Applicants: Krystle PEREZ, Seattle, WA (US); Elena M. BOSQUE, Seattle, WA (US); University of Washington, Seattle, WA (US); Seattle Children’s Research Hospital (DBA Seattle Children’s Research Institute), Seattle, WA (US)

(72) Inventors: Krystle PEREZ, Seattle, WA (US); Elena M. BOSQUE, Seattle, WA (US); Sienna RICE, Seattle, WA (US); Gregory Charles VALENTINE, Seattle, WA (US); Eric J. SEIBEL, Seattle, WA (US); Leonard Y. NELSON, Seattle, WA (US); Jung Woong Samuel SEO, Seattle, WA (US); Benneth SALANGA, Seattle, WA (US); Kathleen KEARNEY, Seattle, WA (US); Liam BURK, Seattle, WA (US); Timothy Rex ROBINSON, Seattle, WA (US)

(73) Assignees: University of Washington, Seattle, WA (US); Seattle Children’s Research Hospital (DBA Seattle Children’s Research Institute), Seattle, WA (US)

(21) Appl. No.: 18/565,075

(22) PCT Filed: May 27, 2022
(86) PCT No.: PCT/US2022/031370

§ 371 Date: Nov. 28, 2023

Related U.S. Application Data
(60) Provisional application No. 63/194,669, filed on May 28, 2021.

(51) Int. Cl. A61M 5/168; A61M 25/02

(52) U.S. Cl. CPC A61M 5/16836; A61M 2025/0266; A61M 2205/3306; A61M 2205/584

(57) ABSTRACT

An example device for detecting intravenous (IV) infiltration by identifying leaks and swelling at the patient skin. The device uses skin swelling to displace alignment of overlapping layers to produce a pattern or indication of swelling. Leakage is detected through the use of a conduit to wick liquid away from an insertion site and react with a dye or reagent to produce a color change that makes a clear visible indication of leaking. The detection methods provide visible markers without occluding visibility of the insertion site for the IV therapy.

### CROSS-REFERENCE TO RELATED APPLICATION(S)

This application claims the priority of U.S. Provisional Application No. 63/194,669, filed on May 28, 2021, and which is incorporated by reference herein in its entirety.

### TECHNICAL FIELD

This application relates to devices, systems, and methods for detecting intravenous (IV) infiltration and leakage around an IV insertion site.

### BACKGROUND

IV infusion therapy is a widespread medical technique in which fluid nutrients or fluid medicaments are infused into the bloodstream of a patient through an IV tube as part of a medical procedure for treatment of the patient. More specifically, an IV administration system for IV infusion includes a fluid source, an IV catheter (referred to as “tube” hereafter) and a venipuncture device (needle) inserted through the skin into one of the patient's blood vessels (e.g., at a venous access site). This establishes a flow path from the fluid source to the blood vessel. The fluid nutrients or medicaments may be pumped from the fluid source through the IV tube or drained by gravity through the IV tube into the patient's bloodstream.

IV Infiltration is a highly common IV therapy complication and is defined as the penetration of IV medications or fluids into the surrounding tissue due to the dislodgement of the catheter or puncturing of the blood vessel. Infiltration directly causes injury to the tissue, creating cellular tissue damage from disrupting cellular transport and impairing cellular function. In this application, we use infiltration to include the terms “extravasation” or “IV failure event”. The main difference between infiltration and extravasation is the type of fluid or IV medication leaking into the surrounding skin or tissue. Herein, we use “infiltration” to refer to all types of fluids or IV medications leaking into the surrounding tissue and causing tissue damage. This tissue damage causes necrosis, amputation, or even death. Newborns are at a higher risk of infiltration and the damages that follow due to their small blood vessel size, fragility of the blood vessels, lack of subcutaneous tissue, and inability to communicate their pain. In low-resource settings, infants often don't have access to devices that could monitor for an IV infiltration, due to a lack of resources. Therefore, healthcare workers manually identify the physical signs of infiltration. In addition to lacking equipment, these regions may also lack neonatal healthcare providers and may have patient to provider ratios of 40:1. Thus, if an infiltration does occur, it can be hours before it is caught. A cost-efficient and resource-conscious solution to detect IV infiltration would have the potential to save lives in many environments. In hospital environments with sophisticated monitoring equipment, monitoring for IV infiltration is a resource intensive task that requires continuous monitoring with sophisticated technologies. Accordingly, a cost-efficient solution that is simple and easy to implement and monitor can improve patient outcomes in any setting.

Infiltration of IV fluid into a patient's tissue can also occur during the course of fluid infusion to a patient even though the IV set was originally established for proper operation. For example, patient motion may cause a needle that was originally properly inserted into a blood vessel to become separated from the vessel or lodge against the wall of the vessel or another obstruction. Moreover, the infusion needle may become clotted or an occlusion may occur in the IV tube upstream of the needle. The following disclosure and claims will be generally couched in terms of an infiltration condition, but the term “infiltration” will be understood to include such conditions as clogging of the needle or occlusion of the tube.

Visual and tactile examinations of IV sites are the most widely used methods for detecting infiltrations. The infiltrated site may appear swollen or puffy. In this case erythema may also be present. Infiltrations may also appear as a pale area where the infiltrate has pooled below the skin. The skin may feel cooler than the surrounding area due to rapid entrance of the IV fluid into the tissue before it can be warmed to body temperature. The visual and tactile examination technique is ineffective in detecting infiltration, since by the time infiltration is detected, tissue damage has already occurred.

### BRIEF DESCRIPTION OF THE DRAWINGS

The detailed description is described with reference to the accompanying figures. In the figures, the left-most digit(s) of a reference number identify the figure in which the reference number first appears.

FIG. 1 illustrates an example of a patient receiving IV infusion therapy with an IV infiltration detection device, according to at least one example.

FIG. 2A illustrates an example IV infusion site with an infiltration detection device in a normal state, according to at least one example.

FIG. 2B illustrates an example IV infusion site with an infiltration detection device in a swollen state, according to at least one example.

FIG. 3A illustrates an example IV infusion site with an IV leak detection device in a normal state, according to at least one example.

FIG. 3B illustrates an example IV infusion site with an IV leak detection device indicating a leak, according to at least one example.

FIG. 4A illustrates an example IV infusion site with an infiltration detection device in a normal state, according to at least one example.

FIG. 4B illustrates an example IV infusion site with an infiltration detection device indicating IV infiltration, according to at least one example.

FIG. 5 illustrates layers of an IV infiltration detection device, according to at least one example.

FIG. 6 illustrates an example of an IV infiltration detection device indicating IV infiltration, according to at least one example.

FIG. 7 illustrates layers of an IV infiltration detection device using pattern overlap, according to at least one example.

FIG. 8 illustrates layers of an IV infiltration detection device using marker lines, according to at least one example.

FIG. 9A illustrates an IV infiltration detection device using pattern overlap indicating a normal state, according to at least one example

FIG. 9B illustrates an IV infiltration detection device using pattern overlap indicating an infiltration, according to at least one example.

FIG. 10A illustrates an IV infiltration detection device using marker lines indicating a normal state, according to at least one example

FIG. 10B illustrates an IV infiltration detection device using marker lines indicating an infiltration, according to at least one example.

### DETAILED DESCRIPTION

Various implementations described herein relate to detection of IV infiltration and leakage. Example systems and methods for IV infiltration include application of a clear, flexible adhesive patch that replaces existing IV securement bandages and/or tapes. Therefore, in various implementations, the adhesive patch secures the IV to the injection site and provides visible inspection of the injections site as well as notification and/or detection of IV infiltration. In particular, various systems and methods described herein detects two symptoms of IV infiltration, swelling caused by pooling fluid in the tissue and fluid leakage out of the injection site. Example systems described herein enable IV infiltration detection in patients, including in infants, in a reliable manner and uses no electricity, which decreases costs and improves utilization in various clinical environments (e.g., low-resource environments).

Example IV infiltration detection systems described herein detect IV leakage and swelling to identify IV infiltration. In some examples, the detection systems include transparent adhesive patches with an opaque border to detect swelling and liquid from fluid back up due to IV infiltration. The term ‘occluded,’ and its equivalents as used herein, may refer to the state of one element being visibly hidden or otherwise overlapped by another element. The swelling mechanism of the adhesive patch senses skin strain from the fluid buildup outside the blood vessel. In some examples, the swelling due to the skin strain causes a sliding mechanism including two strips, one with a socket and one with a colorful tip that is inserted inside the socket so that when combined acts as one strip. The strip is anchored with adhesive at opposite ends of the patch and placed distal to the insertion site. When skin strain is sensed, the two strips slide apart as a result of the skin swelling and strain and the colored portion of the strip is revealed to provide a visible alert of an infiltration.

In some examples, the patch may use other mechanisms to detect and alert to skin swelling. For instance, the sliding portions of the strip may use a Moire or other pattern(s) or shapes, such as geometric patterns, rather than a colored tip and socket configuration. The sliding portions of the strip may be attached at opposite ends of the patch and overlap at a middle portion of the patch with free ends of the sliding strips capable of sliding relative to one another and the underlying adhesive patch. The Moire or geometric pattern may include one or more different patterns on layers capable of sliding with respect to one another that produce different overall patterns based on a swelling state underneath the patch. In a particular example, a patch may include three different layers. A substrate of the patch may adhere to the skin of the patient over the injection site. A first layer attached to the substrate may include a first pattern such as a thin red Moire or geometric pattern. The first layer is anchored at a first edge of the patch with adhesive. A second layer is attached to the substrate at a second edge of the patch opposite the first edge. The second layer has a second pattern, such as a thicker green Moire or geometric pattern that covers the first pattern when overlapped such that the green pattern is only seen during a normal unswollen state. The anchoring opposite one another of the first layer and the second layer enables the first layer and the second layer to slid across each other. A third layer is anchored to both the first side and the second side of the patch to secure the first and second layers to the patch. When swelling occurs under the patch due to IV infiltration, the skin strain causes the Moire or geometric pattern to shift, and reveals the red pattern of the first layer from underneath the green pattern of the second layer, providing a visual indication of IV infiltration.

In another example of swelling detection, the patch includes two layers, a first layer that is stretchable and flexible and will deform and stretch with swelling of the skin and a second layer that is flexible but not stretchable. The second layer includes a brightly colored edge around the perimeter thereof. The second layer is retained in place with the first layer by a retainer sleeve adhered to the flexible layer such that the second layer is able to move independent of the first layer. When swelling occurs, the first layer expands with the swelling skin, but the second layer does not stretch with the strain from the swelling skin and begins to slide out of the retainer sleeve, exposing a brightly colored edge and providing an indication of IV infiltration. This example enables easy detection of swelling with a highly visible indicator and also enables clear and unobstructed visibility of the IV injection site.

In some examples, the patch may include a fluid detection mechanism to detect IV infiltration based on IV leakage. The patch includes a string, wick, or gauze material that is infused with a dye that changes color when in contact with liquid from the IV, such as methylene blue, an FDA approved dye. The string or wick may extend from an edge of the patch to a center of the patch at the center of the injection site when placed on a patient. The string may be infused with the dye at the periphery or adjacent an edge of the patch such that the liquid may be wicked away from the injection site and the dye provide a visible color at the edge thereby providing an unobstructed view of the injection site through the patch. In some examples, such as infants, the patch may be used to identify IV leakage since the string is not exposed to a surrounding environment or other liquid source (since infants minimally sweat, if at all) and therefore the only or primary liquid that the string will absorb is the IV fluid leaking from the injection site. The capillary action of the string wicks the liquid away from the injection site to the edge of the patch where the dye is infused on the string thereby providing the visual indication of IV leakage.

In some examples, a patch may include both swelling and leakage detection to identify IV infiltration. In such examples, the patch may include a swelling detection mechanism such as a geometric pattern including a Moire pattern, colored tip in socket, or sliding pattern mechanisms described above as well as a leakage detection system such as the string and dye mechanism described above. In this manner, the single patch may be capable of detecting IV infiltration through either swelling and/or liquid leakage. Accordingly, the patch can function at various IV injection site locations and also provides an extremely low-cost design that does not rely on any electricity for detection of the IV infiltration.

The systems and methods herein provide several benefits over conventional systems for detecting IV infiltration and leakage. The systems, devices, and methods described herein provide for reliable and efficient detection in a robust manner at low cost. Accordingly, the systems described herein may provide easy detection of infiltration and leakage particularly in understaffed, rural, emergency, and low-resource settings.

Implementations of the present disclosure will now be described with reference to the accompanying figures.

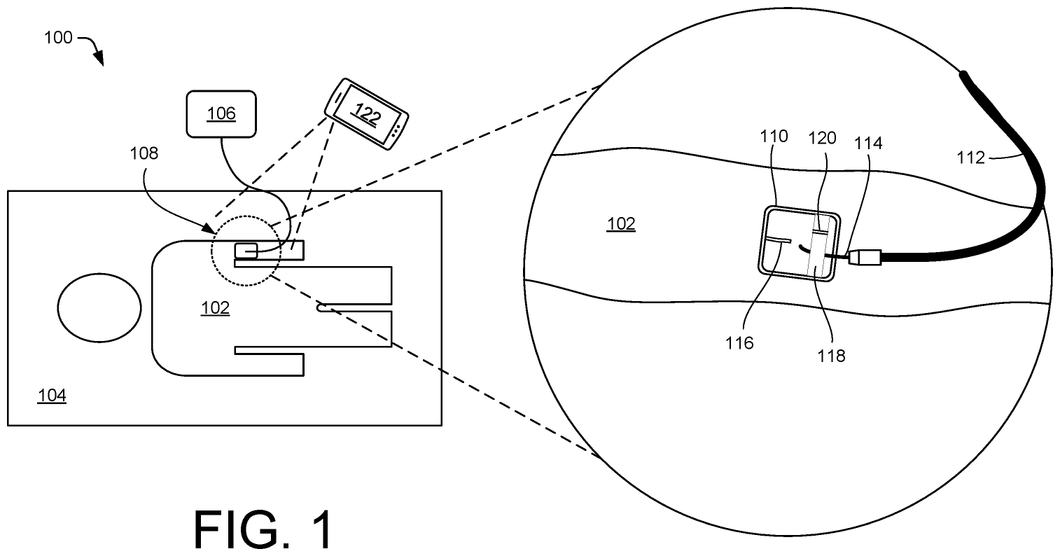

FIG. 1 illustrates an example 100 of a patient 102 receiving IV infusion therapy with a patch 110, according to at least one example. The patient 102 is laying on a bed 104 receiving IV infusion therapy from an IV device 106 via an injection needle 114 and IV tube 112. A detail view 108 of the arm of the patient 102 provides a view of the patch 110 in place on the arm of the patient. The injection needle 114 is held in place by a patch 110 equipped with IV infiltration detection mechanisms to alert a caregiver in the event of IV infiltration. The patch 110 adheres to the skin of the patient 102 and holds the injection needle 114 in place with the adhesive.

The patch 110 includes a leakage detection mechanism 116 and a swelling detection mechanism 118. The leakage detection mechanism 116 and/or the swelling detection mechanism 118 may identify IV infiltration by identifying symptoms of IV infiltration. The detection mechanisms are highly visible and therefore can provide immediate indication of IV infiltration before damage occurs to the skin of the patient. Additionally, the patch 110 is transparent and therefore also enables visual inspection and monitoring of the injection site during use.

The leakage detection mechanism 116 detects the presence of liquid in the environment underneath the patch 110, between the patch 110 and the skin of the patient 102. The patch 110 may form an impermeable barrier against liquid, therefore preventing liquid from entering into the space under the patch 110. In some examples, the patch 110 may be formed of a polyurethane membrane coated with a layer of an acrylic adhesive. In some examples, the patch 110 may be formed of other polymers, films, and materials. In some examples, the patient 102 may not sweat, thereby eliminating an additional source of potential liquid. For instance, the patient 102 may be an infant. Accordingly, any liquid in the space between the patch 110 and the patient 102 may be assumed to be a result of leakage from the injection needle 114 at the injection site. The leakage detection mechanism 116 therefore can be used to detect the presence of liquid in the space between the patch 110 and the skin of the patient 102.

The patch 110 includes a string, wick, or gauze material as part of the leakage detection mechanism 116. The string or other moisture-wicking material is infused with a dye or compound that changes color when in contact with liquid from the IV, such as methylene blue, a US Food and Drug Administration (FDA) approved dye. In some examples, particular dyes or compounds may be selected that create visible color changes when reacting with particular compounds of the IV infusion therapy, such as by reacting with a particular medication provided to the user through the injection needle. In this manner, the leakage detection mechanism 116 may identify leaks of the particular compound but may be prevented from identifying false positives from skin sweat or other potential sources of liquids. The string of the leakage detection mechanism 116 extends from an edge of the patch 110 to a center of the patch 110 that may be placed over the injection site. The string may be infused with the dye at the periphery of the patch 110 or adjacent an edge of the patch 110 such that the liquid detected by the leakage detection mechanism 116 is wicked away by the moisture wicking material forming the string and provide a visible indication of leakage at a location away from the injection site. In this manner, the view of the injection site through the patch 110 may remain unobstructed for visual inspection and monitoring. The capillary action of the string (e.g., due to the moisture-wicking behavior) wicks the liquid away from the injection site to the edge of the patch 110 where the dye is infused on the string, thereby providing the visual indication of IV leakage when the liquid reaches the dye on the string.

The swelling detection mechanism 118 detects skin strain from the fluid buildup outside the blood vessel as a result of IV infiltration. In the example shown in FIG. 1, the swelling detection mechanism includes a sliding mechanism including two strips, one including a socket to receive the other. The strip that is received by the socket includes a colorful tip that fits within the socket and is not visible when the two strips are in a first position. The sliding strips are anchored with adhesive at opposite ends of the patch 110. When skin strain occurs as a result of IV infiltration, the swelling creates upward pressure on the patch 110 and causes the two strips to slide apart and reveal the colored indicator 120 as a visible alert of IV infiltration.

In some examples, the leakage detection mechanism 116 and/or swelling detection mechanism 118 may be detectable by a computing device, such as through the use of a user device 122 equipped with and/or in communication with an imaging device. The user device 122 may capture image data (e.g., a digital image) of the patch 110 and may identify or detect swelling and/or leakage based on the leakage detection mechanism 116 and/or the swelling detection mechanism 118. For example, the swelling detection mechanism 118 may be evaluated by the user device 122 to identify an amount of swelling as a function of the proportion of the colored indicator 120 visible outside of the socket as described above. The user device 122 may be used to identify when swelling is outside of an acceptable threshold amount and alert to swelling. In some examples, the user device 122 may be more sensitive to swelling detection than a binary indication of swelling or not swelling that may be provided by some mechanisms described herein.

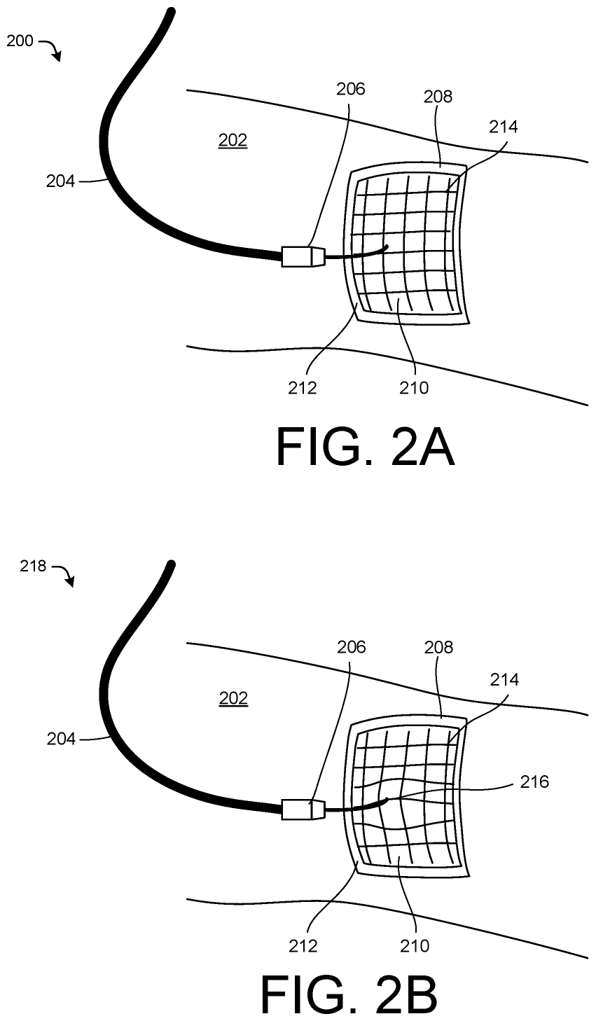

FIG. 2A illustrates an example IV infusion site 200 with an infiltration detection device 208 in a normal state, according to at least one example. The infiltration detection device 208 is placed on a patient 202 over an injection site where a needle 206 is used to provide IV infusion therapy to provide a liquid via a tube 204 to the patient 202. The infiltration detection device 208 includes a transparent patch 210 that adheres to the skin of the patient 202. In some examples, the transparent patch 210 may have an adhesive 212 around a perimeter of the transparent patch 210 to secure to the patient 202. In some examples, the adhesive 212 may cover an entire surface of the transparent patch 210 to secure to the patient 202. The transparent patch 210 is formed of a flexible material that is also capable of stretching in at least two directions, along directions parallel with the surface of the transparent patch 210.

The infiltration detection device 208 includes a pattern 214 of lines on the surface of the transparent patch 210. The pattern 214 includes a first set of parallel lines in a first direction and a second set of parallel lines perpendicular to the first set of lines. The pattern 214 provides a visual indication of the contours of the skin of the patient 202 and can be used to identify changes in the contours of the skin to identify swelling of the skin that may be a result of IV infiltration. Though depicted with a particular pattern of lines, additional patterns may be used that provide indications of the contours of the skin underlying the infiltration detection device 208.

FIG. 2B illustrates the example IV infusion site 218 which may be the example IV infusion site 200 of FIG. 2A with an infiltration detection device 208 in a swollen state, according to at least one example. In the example IV infusion site 200 as depicted in FIG. 2B, the patient 202 is experiencing swelling 216 due to IV infiltration at the injection site. The swelling 216 causes the infiltration detection device 208 to change shape due to the changing shape of the skin of the patient 202 and also causes stretching and deformation of the infiltration detection device 208 due to the flexible and stretchable nature of the material of the transparent patch 210. According, as the infiltration detection device 208 changes shape while the skin swells, the pattern 214 changes shape to produce a different pattern. The swelling 216 may be easily identified by observing (e.g., visually observing) the change in the pattern on the infiltration detection device 208.

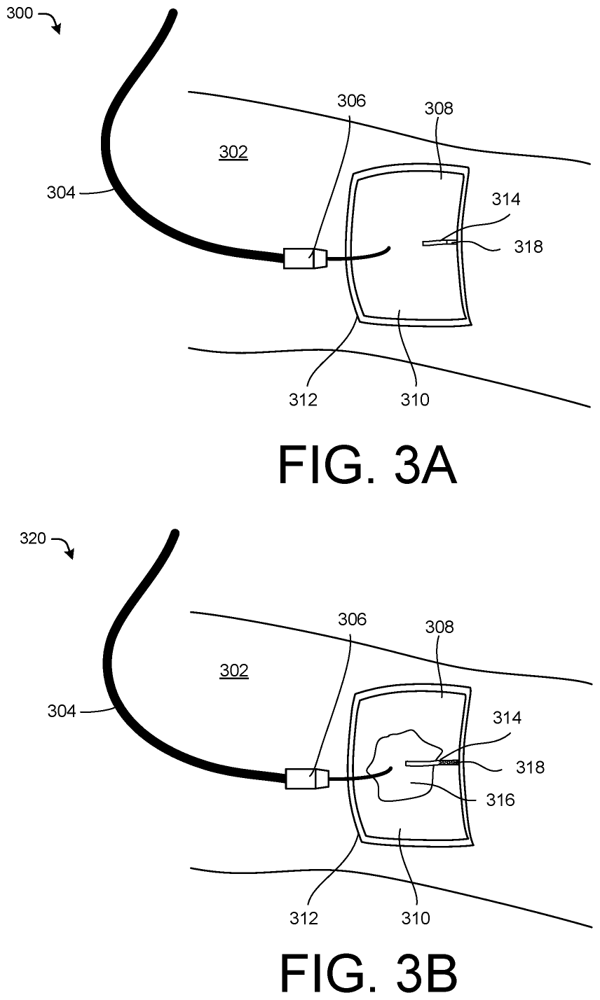

FIG. 3A illustrates an example IV infusion site 300 with an infiltration detection device 308 in a normal state, according to at least one example. The infiltration detection device 308 is placed on a patient 302 over an injection site where a needle 306 is used to provide IV infusion therapy to provide a liquid via a tube 304 to the patient 302. The infiltration detection device 308 includes a transparent patch 310 that adheres to the skin of the patient 302. In some examples, the transparent patch 310 may have an adhesive 312 around a perimeter of the transparent patch 310 to secure to the patient 302. In some examples, the adhesive 312 may cover an entire surface of the transparent patch 310 to secure to the patient 302.

The infiltration detection device 308 includes a conduit 314 for wicking liquid away from the injection site, positioned at a center portion of the infiltration detection device 308, towards an edge of the infiltration detection device 308. The conduit 314 may include a liquid wicking material such as a string, gauze, fiber, paper, or other such material. The conduit 314 may also include a tube or passage that uses capillary action to transport a liquid towards the edge of the infiltration detection device 308. The conduit 314 may include or may be in communication with a dye region 318. The dye region 318 may include a material infused with a dye, a collection of a dye material, or other compound that changes color when mixed with a liquid. In a particular example, the conduit 314 may include a string that acts as a wick while a portion of the string adjacent an edge of the infiltration detection device 308 is infused with methylene blue. The methylene blue will change color when liquid comes in contact with the dye. Methylene blue is hydrophilic in addition to being non-irritating for the skin of the patient 302 and being non-staining and biocompatible. Such attributes make a dye such as methylene blue especially well suited for indication of a liquid. When dry, the dye is a first color, such as colorless or virtually colorless while the same dye, when wet, is a highly visible color, such as a deep blue that is easily detectable by a caregiver.

In some examples, the dye may be selected or intended for use with particular IV infusion therapies. For example, a particular therapy may include an IV liquid that includes a particular active component or compound. A dye may be selected that reacts with the particular compound or active component of the liquid. In this manner, the infiltration detection device 308 may detect liquid as a result of infiltration of the IV liquid but not trigger false positives as a result of sweat or other liquids that may happen to reach the conduit 314.

In some examples, the dye region 318 may include a dye packet that stores a color changing material. The dye packet may be encapsulated to insulate from sweat or other liquids and only be reachable through the conduit 314 by capillary along the conduit. The dye packet may include a collection of a dry or liquid dye that reacts with the presence of a particular compound or with the presence of moisture to result in a color change.

In some examples, the dye region 318 may include a perimeter or a portion of the perimeter of the infiltration detection device 308. The conduit 314 provides an avenue to transporting leaking liquid away from the injection site to provide the visible indicator such that the indicator does not obstruct the view of the injection site. In some examples, the dye region 318 may include a gauze band or strip around a perimeter of the infiltration detection device 308.

FIG. 3B illustrates an example IV infusion site 320 that may be the example IV infusion site 300 of FIG. 3A showing an infiltration detection device 308 indicating a leak, according to at least one example. The example IV infusion site 320 may be experiencing IV infiltration and as a result may be leaking a liquid 316. The liquid 316 is transported by the conduit 314 to the dye region 318 where the dye region 318 changes color to indicate the leak. The visible color change is readily apparent to a caregiver without careful examination being required.

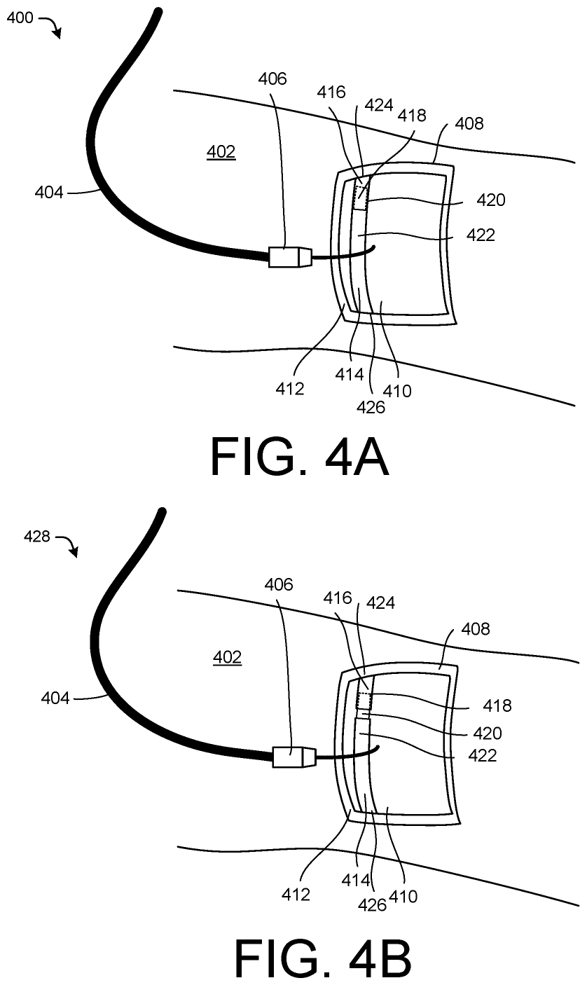

FIG. 4A illustrates an example IV infusion site 400 with an infiltration detection device 408 in a normal state, according to at least one example. The infiltration detection device 408 is placed on a patient 402 over an injection site where a needle 406 is used to provide IV infusion therapy to provide a liquid via a tube 404 to the patient 402. The infiltration detection device 408 includes a transparent patch 410 that adheres to the skin of the patient 402 and provides an unobstructed view of the injection site. In some examples, the transparent patch 410 may have an adhesive 412 around a perimeter of the transparent patch 410 to secure to the patient 402. In some examples, the adhesive 412 may cover an entire surface of the transparent patch 410 to secure to the patient 402.

The infiltration detection device 408 includes an indicator strip 414 that provides a visible indication of swelling to indicate potential IV infiltration to a caregiver for treatment or remediation. The indicator strip 414 detects skin strain from the fluid buildup outside the blood vessel as a result of IV infiltration. The indicator strip 414 includes a first portion 416 and a second portion 422 that are slidably coupled together. The first portion 416 includes a receiving end that defines a socket 418 to receive a tip 420 of the second portion 422. The socket 418 enables the tip 420 of the second portion 422 to slide relative to the first portion and reveal a portion of the tip 420 in some situations. The socket 418 may include any opening or covering portion that receives the tip 420 and obscures view of the tip 420 until the tip 420 is slid as a result of relative motion of the first portion 416 and the second portion 422.

The first portion 416 is adhered or coupled to the transparent patch 410 at a first end 424 that is opposite the socket 418. The second portion 422 is adhered or coupled to the transparent patch 410 at a second end 426 opposite the tip 420. The first portion 416 and second portion 422 may therefore move relative to one another at a middle section of the indicator strip 414.

The tip 420 includes a visible indicator, such as a brightly colored, colorful, patterned, reflective, fluorescent, or other such visible applications that make the tip 420 easily identifiable when visible. During a normal state, such as when the skin of the patient 402 is not swollen, the tip 420 remains seated within the socket 418 such that the visible indicator of the tip 420 is not visible, indicating normal conditions to a caregiver.

FIG. 4B illustrates an example IV infusion site 428 with an infiltration detection device 408 indicating IV infiltration, according to at least one example. The example IV infusion site 428 includes the elements described above with respect to FIG. 4A. During IV infiltration, the skin of the patient 402 may swell, the swelling causing a strain on the infiltration detection device 408. The indicator strip 414 will move as a result of the skin strain from the fluid buildup outside the blood vessel. In particular, the indicator strip 414 will elongate as a result of the swelling. The elongation of the indicator strip 414 is enables by the slidable coupling of the first portion 416 and the second portion 422.

As the indicator strip 414 elongates, the tip 420 slides out of the socket 418, revealing the visible indicator on the tip 420 for view by a caregiver. The visible indicator therefore provides a clear visible indication of swelling that may not be detectable without significant additional observation. The indicator strip therefore provides a rapid and effective manner of highlighting swelling and alerting a caregiver of potential IV infiltration. The indicator strip 414 is shown in a first orientation in FIG. 4, but may be positioned and/or oriented otherwise with respect to the transparent patch 410 in other examples.

FIG. 5 illustrates layers of an IV infiltration detection device 500, according to at least one example. The IV infiltration detection device 500 includes a first layer 502, a second layer 504, and a retainer 510. The first layer 502 is a transparent layer that may be formed of a typical bandage material for securing and IV needle, such as a dressing tape that is transparent. The first layer 502 is flexible and stretchable so it can deform and stretch with changes in the shape of the skin of the patient, for example as swelling occurs. A first side (e.g., a patient-facing side) of the first layer includes a pressure sensitive adhesive for securing to the patient. In some examples, the first layer may be formed of a polyurethane film with the first side coated with an acrylic adhesive. The polyurethane film provides a barrier against water vapor, oxygen, and organisms.

The second layer 504 is flexible such that it is capable of conforming to the shape of the skin when applied to the patient, but is not stretchable. Such examples of these layers include, but are not limited to polyurethane or PET (polyethylene terephthalate). The second layer 504 includes a transparent portion 506 at a middle of the second layer that enables visibility through the IV infiltration detection device 500. The second layer 504 also includes a border 508 that includes a visible marker such as a pattern or colorful portion that is highly visible to a caregiver. The border 508 may include printed, painted, and otherwise formed visible indicators. The border may include colors, patterns, fluorescent dyes, reflective, or other visible markers.

The retainer 510 adheres to the first layer 502 to slidably secure the second layer 504 to the first layer 502. The retainer 510 may serve as a retainer ring that is fixed to the first layer with the second layer 504 sandwiched between the first layer 502 and the retainer 510. The retainer 510 includes an opening that has a size and shape that correspond to the size and shape of the transparent portion 506 of the second layer 504. The retainer 510 is opaque to prevent visibility of the border 508 when the border is positioned behind the retainer 510. Therefore, when assembled together, the retainer 510 obstructs the view of the border 508 of the second layer 504. The retainer 510 may be formed of the same material as the first layer 502 and may be flexible and stretchable with the first layer 502. The second layer 504 may be free to move within the frame of the retainer 510. In some examples, the second layer 504 has a surface area smaller than a surface area of the first layer 502 such that the second layer 504 can be slidably held in place by the retainer 510.

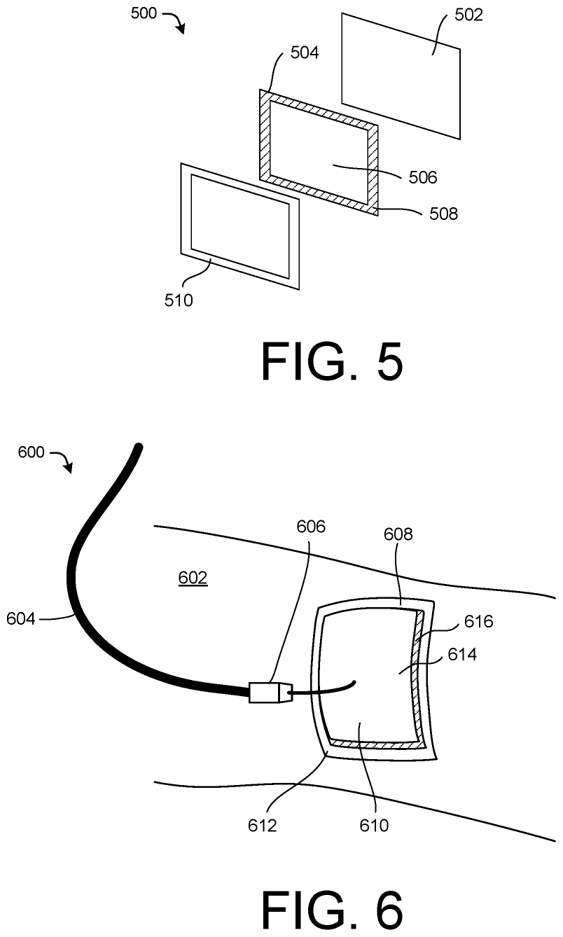

FIG. 6 illustrates an example of an IV infiltration detection device 608 indicating IV infiltration secured to a patient 602, according to at least one example. The example of FIG. 6 includes an IV needle 606 and tube 604 that provide IV fluid for an IV therapy treatment. The IV infiltration detection device 608 may be an example of the IV infiltration detection device 500 of FIG. 5. Accordingly, the IV infiltration detection device 608 may include a first layer 610, a second layer 614 including a border 616, and a retainer 612 that may correspond to the first layer 502, the second layer 405, the border 508, and the retainer 510 of FIG. 5.

In the example of FIG. 6, when swelling occurs, the first layer 610 of the IV infiltration detection device 608 may expand and stretch with the swelling of the skin, in addition to conforming to the shape of the skin of the patient 602 when applied. The retainer 612 also stretches and flexes to conform with the shape of the skin and stretches with the swelling of the skin. The second layer 614, being flexible so as to conform with the shape of the patient but not stretchable, therefore will have a different shape and/or size than the first layer 610 and/or the retainer 612 when swelling occurs. The difference in size and/or shape between the first layer 610 and/or the retainer 612 and the second layer 614 results in the border 616 being visible as it protrudes from behind the retainer 612. The visibility of the border 616 provides an indication of swelling at the patient that must be addressed.

In some examples, the second layer 614 may be secured to an edge of the first layer 610 and/or the retainer 612 such that a portion of the second layer 614 moves with the first layer 610 and/or the retainer 612 while a second free portion of the second layer 614 is free to move independent of the first layer 610 and/or the retainer 612.

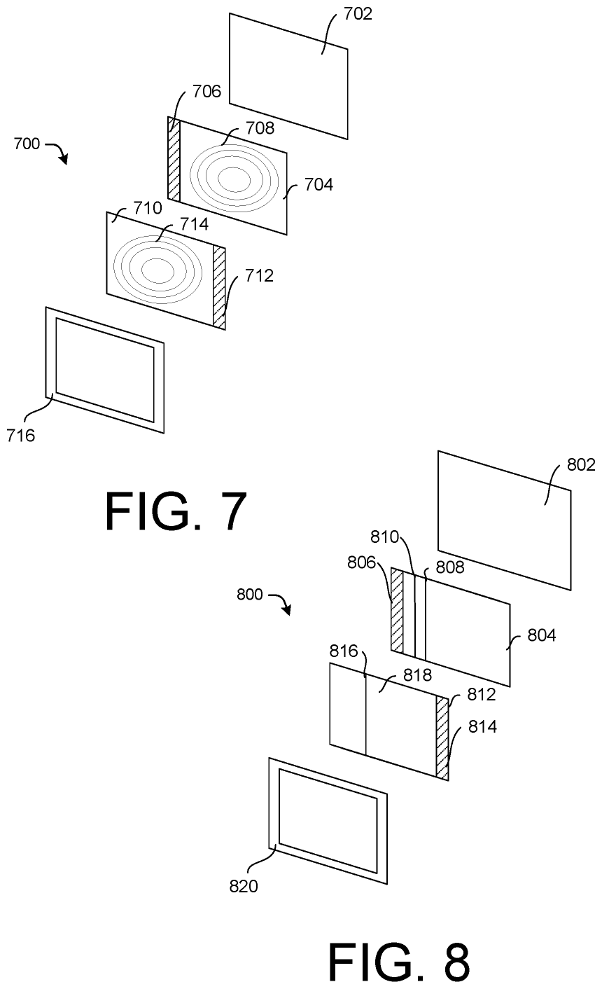

FIG. 7 illustrates layers of an IV infiltration detection device 700 using pattern overlap, according to at least one example. The IV infiltration detection device 700 includes a first layer 702, a second layer 704, a third layer 710, and a retainer 716. The first layer 702 is a transparent layer that may be formed of a typical bandage material for securing and IV needle, such as a dressing tape that is transparent. The first layer 702 is flexible and stretchable so it can deform and stretch with changes in the shape of the skin of the patient, for example as swelling occurs. A first side (e.g., a patient-facing side) of the first layer 702 includes a pressure sensitive adhesive for securing to the patient.

The second layer 704 is flexible such that it is capable of conforming to the shape of the skin when applied to the patient, but is not stretchable. The second layer 704 is transparent and includes a pattern 708 that may be transparent, semi-transparent, or opaque at a middle of the second layer 704. The pattern 708 does not occlude the visibility through the IV infiltration detection device 700. The pattern 708 may be a first pattern that forms a Moire pattern when combined with a pattern 714 on the third layer 710. The second layer 704 is secured to the first layer with a pressure sensitive adhesive or other adhesive at one end 706 such that the end 706 is fixed to the first layer 702 but the opposite end is free to move relative to the first layer 702. The pattern 708 may include printed, painted, and otherwise formed visible indicators.

The third layer 710 is flexible such that it is capable of conforming to the shape of the skin when applied to the patient, but is not stretchable. The third layer 710 is transparent and includes a pattern 714 that may be transparent, semi-transparent, or opaque at a middle of the third layer 710. The pattern 714 does not occlude the visibility through the IV infiltration detection device 700. The pattern 714 may be a first pattern that forms a Moire pattern when combined with the pattern 708 on the second layer 704. The third layer 710 is secured to the first layer 702 with a pressure sensitive adhesive or other adhesive at one end 712 such that the end 712 is fixed to the first layer 702 but the opposite end of the third layer 710 is free to move relative to the first layer 702 and the second layer 704. The pattern 714 may include printed, painted, and otherwise formed visible indicators.

The retainer 716 adheres to the first layer 702 to slidably secure the second layer 704 and the third layer 710 to the first layer 702. The retainer 716 includes an opening that has a size and shape that corresponds to a size and shape of the patterns 708 and 714 and/or of a transparent portion of the IV infiltration detection device 700. The retainer 716 may be formed of the same material as the first layer 702 and may be flexible and stretchable with the first layer 702. The second layer 704 and third layer 710 may be free to move within the frame of the retainer 716.

As swelling occurs on the skin of the patient, the patterns 708 and 714 will shift relative to one another due to being fixed to different portions of the first layer 702 and therefore the resulting overlapped pattern will provide a different appearance. In some examples, the pattern 714 may be opaque and may match the pattern 708 such that in a normal condition the pattern 708 is obstructed by the pattern 714. During swelling, the pattern 708 and the pattern 714 become misaligned and therefore the pattern 708 becomes visible.

In some examples, the patterns 708 and 714 may be formed of a conductive wire, and/or the second layer and third layer may include a conductive and/or capacitive element that may be used to identify misalignment of the layers that results from swelling underneath the IV infiltration detection device 700. For instance, a first element on the second layer 704 may be electrically coupled to a monitoring device that monitors a capacitance of the first element as a result of alignment with a second element positioned on the third layer 710. The first element and/or second element may include a coil or other electronic device to produce an electric field that may be detected by the other of the first element or the second element. The strength of the electric field may be used to identify or determine the alignment of the second layer 704 and the third layer 710. Misalignment of the layers is a result of swelling as described herein and may be identified by the monitoring device that may output an alert if misalignment over a threshold amount is identified that may correspond to a swelling of the skin underneath the IV infiltration detection device 700.

FIG. 8 illustrates layers of an IV infiltration detection device 800 using marker lines, according to at least one example. The IV infiltration detection device 800 includes a first layer 802, a second layer 804, a third layer 812, and a retainer 820. The first layer 802 is a transparent layer that may be formed of a typical bandage material for securing and IV needle, such as a dressing tape that is transparent. The first layer 802 is flexible and stretchable so it can deform and stretch with changes in the shape of the skin of the patient, for example as swelling occurs. A first side (e.g., a patient-facing side) of the first layer 802 includes a pressure sensitive adhesive for securing to the patient.

The second layer 804 is flexible such that it is capable of conforming to the shape of the skin when applied to the patient, but is not stretchable. The second layer 804 is transparent and includes a first marker 808 and a second marker 810 that may be transparent, semi-transparent, or opaque. The first marker 808 and/or the second marker 810 may not occlude the visibility through the IV infiltration detection device 800. The second layer 804 is secured to the first layer with a pressure sensitive adhesive or other adhesive at one end 806 such that the end 806 is fixed to the first layer 802 but the opposite end is free to move relative to the first layer 802.

The third layer 812 is flexible such that it is capable of conforming to the shape of the skin when applied to the patient, but is not stretchable. The third layer 812 is transparent at a middle portion 818 and includes a marker 816 that is opaque to occlude one of the first marker 808 and/or the second marker 810. The third layer 812 is secured to the first layer 802 with a pressure sensitive adhesive or other adhesive at one end 814 such that the end 814 is fixed to the first layer 802 but the opposite end of the third layer 812 is free to move relative to the first layer 802 and the second layer 804.

The retainer 820 adheres to the first layer 802 to slidably secure the second layer 804 and the third layer 812 to the first layer 802. The retainer 820 includes an opening such that the injection site for the IV treatment is visible through the IV infiltration detection device 800. The retainer 820 may be formed of the same material as the first layer 802 and may be flexible and stretchable with the first layer 802. The second layer 804 and third layer 812 may be free to move within the frame of the retainer 820.

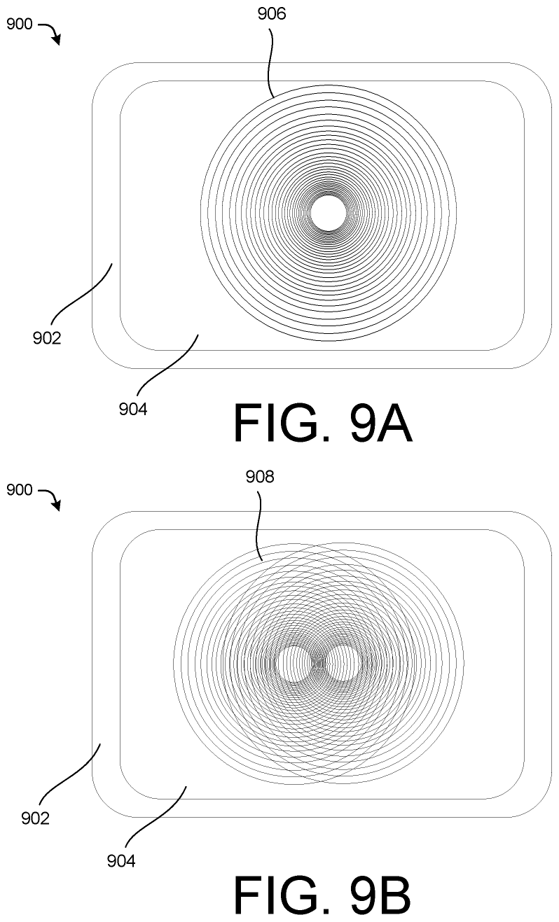

FIGS. 9A and 9B illustrate an IV infiltration detection device 900 using pattern overlap, according to at least one example. The IV infiltration detection device 900 is an example of the IV infiltration detection device 700 of FIG. 7 and includes the components described with respect to FIG. 7. In particular, the IV infiltration detection device 900 includes a border 902 that corresponds to the retainer 716 of FIG. 7 and a transparent portion 904 that provides visibility through the layers of the IV infiltration detection device 900 to the injection site. The IV infiltration detection device also includes the patterns as described above that produce a pattern 906 when placed on the skin of the patient. The pattern 906 shows the aligned layers of the second and third layers as described herein. After application, when swelling occurs in the patient, the pattern 908 shown in FIG. 9B is displayed. The misalignment of the layers as described above produces the pattern 908 that provides a visible indication of misalignment for a caregiver to identify swelling occurring in the patient at the insertion site. The patterns 906 and 908 are illustrations of potential Moire or geometric patterns that may be implemented to identify the misalignment of the layers as a result of swelling.

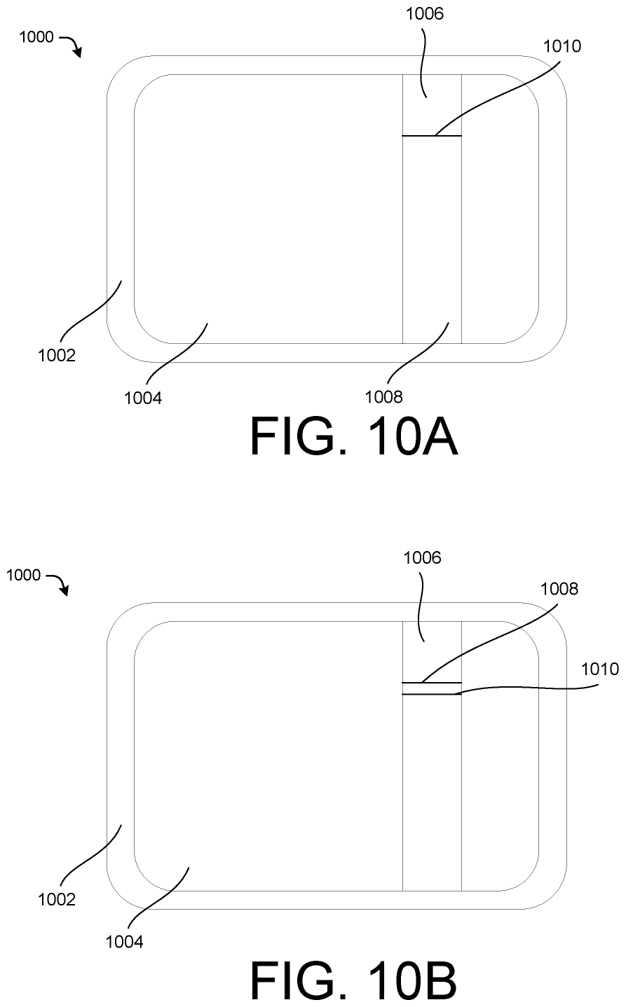

FIGS. 10A and 10B illustrate an IV infiltration detection device 1000 using marker lines, as described with respect to the IV infiltration detection device 800 of FIG. 8, according to at least one example. The IV infiltration detection device 1000 is an example of the IV infiltration detection device 800 of FIG. 8 and includes the components described with respect to FIG. 8. The indicator strip 1006 only occupies a portion of the width of the IV infiltration detection device 1000 in contrast with the example shown in FIG. 8, therefore providing clearer visibility to the insertion site as only the first layer is between the caregiver and the insertion site. The IV infiltration detection device 1000 includes a border 1002 that corresponds to the retainer 820, a transparent portion 1004 that provides visibility to the insertion site. The indicator strip 1006 includes a first marker 1008 and a second marker 1010. In FIG. 10A, before any swelling occurs, the first marker 1008 is occluded by a portion of the third layer, as described with respect to FIG. 8. After swelling has occurred, as pictured in FIG. 10B, the first marker 1008 protrudes around the occluding portion to provide a second line that makes a visible indication to a caregiver of swelling.

### Example Clauses

A. An intravenous (IV) infiltration detection system, including: a first indicator strip including: a first flexible material; a first adhesive disposed on a first end of the first flexible material and configured to secure to skin of a patient; and a visible indicator disposed on a second end of the first flexible material; and a second indicator strip including: a second flexible material; a second adhesive disposed on a first end of the second flexible material, wherein a second end of the second flexible material defines a socket configured to slidably couple with the visible indicator of the first indicator strip and reveal the visible indicator in response to swelling of the skin of the patient.

B. The IV infiltration detection system of clause A, wherein the socket includes an opaque material that occludes the visible indicator when the visible indicator is positioned within the socket.

C. The IV infiltration detection system of clause A or B, wherein the first flexible material and the second flexible material have a first color and the visible indicator includes a second color.

D. The IV infiltration detection system of any one of clauses A to C, wherein the visible indicator includes a reflective or fluorescent coating.

E. The IV infiltration detection system of any one of clauses A to D, wherein the visible indicator includes: a first line visible during a non-swelling state of the skin of the patient; and a second line visible in response to swelling of the skin of the patient.

F. An intravenous (IV) infiltration detection system, including: a first sheet having a first end and a second end and including a flexible material, wherein: the first end of the first sheet includes a first adhesive to secure to skin of a patient; and the second end of the first sheet includes a first visible indicator; and a second sheet having a first end and a second end, wherein: the second end of the second sheet includes a second visible indicator; the second end of the first sheet overlaps the second end of the second sheet; in a first state the first visible indicator and second visible indicator produce a first pattern; and in a second state the first visible indicator and second visible indicator produce a second pattern.

G. The IV infiltration detection system of clause F, wherein the first pattern includes alignment of the first visible indicator and the second visible indicator and the second pattern includes an interference pattern.

H. The IV infiltration detection system of clause F or G, wherein the first state includes a non-swollen state and the second state includes a swollen state.

I. The IV infiltration detection system of any one of clauses F to H, wherein the first sheet includes a transparent material and the second sheet includes a transparent material.

J. The IV infiltration detection system of any one of clauses F to I, wherein the first visible indicator is transparent and includes a first color and the second visible indicator is transparent and includes a second color.

K. The IV infiltration detection system of any one of clauses F to J, wherein the first visible indicator is opaque and the second visible indicator includes a color.

L. The IV infiltration detection system of any one of clauses F to K, wherein the first pattern or the second pattern include a Moire or a geometric pattern.

M. The IV infiltration detection system of any one of clauses F to L, further including: an imaging device configured to capture image data of the first pattern and the second pattern; and a processor configured to: receive the image data from the imaging device; and determine a swelling state associated with the patient based on the image data.

N. An Intravenous (IV) infiltration detection system, including: a first layer having a first side and a second side, wherein: the first side of the first layer has an adhesive disposed thereon for securing to skin of a patient; and the first layer includes a visible border around an area of the second side of the first layer; a retainer adhered to the second side of the first layer; a second layer slidably coupled with the retainer, wherein: the second layer includes an opaque border configured to align with the visible border of the first layer; in a first state, the visible border is occluded by the opaque border; and in a second state the visible border is at least partially visible around the opaque border.

O. The IV infiltration detection system of clause N, wherein: a first middle portion of the first layer is transparent; and a second middle portion of the second layer is transparent.

P. The IV infiltration detection system of clause N or O, wherein the first state includes a non-swollen state and the second state includes a swollen state.

Q. The IV infiltration detection system of any one of clauses N to P, wherein the first layer includes a flexible and stretchable material.

R. The IV infiltration detection system of any one of clauses N to Q, wherein the second layer includes a flexible material.

S. The IV infiltration detection system of any of any one of clauses N to R, wherein the visible border includes a reflective or fluorescent coating.

T. The IV infiltration detection system of any one of clauses N to S, wherein the visible border includes a first color.

U. The IV infiltration detection system of any one of clauses N to T, wherein the visible border includes a third layer adhered to the first layer.

V. An Intravenous (IV) infiltration detection system, including: a patch having a first side and a second side, with an adhesive disposed on a perimeter of the second side, the adhesive configured to secure the patch to skin of a patient around an IV insertion site; and an absorbent material coupled to the first side of the patch, the absorbent material impregnated with a dye.

W. The IV infiltration detection system of clause V, wherein the dye includes methylene blue.

X. The IV infiltration detection system of clause V or W, wherein the patch includes a transparent center portion configured to enable view of an IV insertion site.

Y. The IV infiltration detection system of any one of clauses V to X, wherein the absorbent material includes a moisture-wicking material that extends from a center portion of the patch to an edge of the patch.

Z. The IV infiltration detection system of any one of clauses V to Y, wherein the absorbent material is impregnated with the dye at the edge of the patch.

AA. The IV infiltration detection system of any one of clauses V to Z, wherein the absorbent material is disposed around a perimeter of the first side of the patch.

AB. The IV infiltration detection system of any one of clauses V to AA, wherein the patch further includes a first visible indicator, the IV infiltration detection system further including: a second patch disposed on the second side of the patch, wherein: the second patch includes a second visible indicator; in a first state the first visible indicator and second visible indicator produce a first pattern; and in a second state the first visible indicator and second visible indicator produce a second pattern.

AC. The IV infiltration detection system of any one of clauses V to AB, further including: a first indicator strip formed of a flexible material, the first indicator strip including: a first adhesive disposed on a first end of the first indicator strip; and a visible indicator disposed on a second end of the first indicator strip; and a second indicator strip formed of a flexible material, the second indicator strip including: a second adhesive disposed on a first end of the second indicator strip, wherein a second end of the second indicator strip defines a socket configured to slidably couple with the visible indicator of the first indicator strip and reveal the visible indicator in response to swelling of the skin of the patient.

AD. The IV infiltration detection system of any one of clauses V to AC, wherein the second side of the patch includes a visible border around an area of the patch and the patch further includes: a retainer adhered to the second side of the patch; a layer slidably coupled with the retainer, wherein: the layer includes an opaque border configured to align with the visible border of the patch; in a first state, the visible border is occluded by the opaque border; and in a second state the visible border is at least partially visible around the opaque border.

While the example clauses described above are described with respect to one particular implementation, it should be understood that, in the context of this document, the content of the example clauses can also be implemented via a method, device, system, computer-readable medium, and/or another implementation. Additionally, any of examples A-AD may be implemented alone or in combination with any other one or more of the examples A-AD.

### CONCLUSION

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. An intravenous (IV) infiltration detection system, comprising:
a first indicator strip comprising:
a first flexible material;
a first adhesive disposed on a first end of the first flexible material and configured to secure to skin of a patient; and
a visible indicator disposed on a second end of the first flexible material; and
a second indicator strip comprising:
a second flexible material;
a second adhesive disposed on a first end of the second flexible material,
wherein a second end of the second flexible material defines a socket configured to slidably couple with the visible indicator of the first indicator strip and reveal the visible indicator in response to swelling of the skin of the patient.

2. The IV infiltration detection system of claim 1, wherein the socket comprises an opaque material that occludes the visible indicator when the visible indicator is positioned within the socket.

3. The IV infiltration detection system of claim 1, wherein the first flexible material and the second flexible material have a first color and the visible indicator comprises a second color.

4. The IV infiltration detection system of claim 1, wherein the visible indicator comprises a reflective or fluorescent coating.

5. The IV infiltration detection system of claim 1, wherein the visible indicator comprises:
a first line visible during a non-swelling state of the skin of the patient; and
a second line visible in response to swelling of the skin of the patient.

6. An intravenous (IV) infiltration detection system, comprising:
a first sheet having a first end and a second end and comprising a flexible material, wherein:
the first end of the first sheet comprises a first adhesive to secure to skin of a patient; and
the second end of the first sheet comprises a first visible indicator; and
a second sheet having a first end and a second end, wherein:
the second end of the second sheet comprises a second visible indicator;
the second end of the first sheet overlaps the second end of the second sheet;
in a first state the first visible indicator and second visible indicator produce a first pattern; and
in a second state the first visible indicator and second visible indicator produce a second pattern.

7. The IV infiltration detection system of claim 6, wherein the first pattern comprises alignment of the first visible indicator and the second visible indicator and the second pattern comprises an interference pattern.

8. The IV infiltration detection system of claim 6, wherein the first state comprises a non-swollen state and the second state comprises a swollen state.

9. The IV infiltration detection system of claim 6, wherein the first sheet comprises a transparent material and the second sheet comprises a transparent material.

10. The IV infiltration detection system of claim 9, wherein the first visible indicator is transparent and comprises a first color and the second visible indicator is transparent and comprises a second color.

11. The IV infiltration detection system of claim 6, wherein the first visible indicator is opaque and the second visible indicator comprises a color.

12. The IV infiltration detection system of claim 6, wherein the first pattern or the second pattern comprise a Moire or a geometric pattern.

13. The IV infiltration detection system of claim 6, further comprising:
an imaging device configured to capture image data of the first pattern and the second pattern; and
a processor configured to:
receive the image data from the imaging device; and
determine a swelling state associated with the patient based on the image data.

14. An Intravenous (IV) infiltration detection system, comprising:
a first layer having a first side and a second side, wherein:
the first side of the first layer has an adhesive disposed thereon for securing to skin of a patient; and
the first layer comprises a visible border around an area of the second side of the first layer;
a retainer adhered to the second side of the first layer;
a second layer slidably coupled with the retainer, wherein:
the second layer comprises an opaque border configured to align with the visible border of the first layer;
in a first state, the visible border is occluded by the opaque border; and
in a second state the visible border is at least partially visible around the opaque border.

15. The IV infiltration detection system of claim 14, wherein:
a first middle portion of the first layer is transparent; and
a second middle portion of the second layer is transparent.

16. The IV infiltration detection system of claim 14, wherein the first state comprises a non-swollen state and the second state comprises a swollen state.

17. The IV infiltration detection system of claim 14, wherein the first layer comprises a flexible and stretchable material.

18. The IV infiltration detection system of claim 17, wherein the second layer comprises a flexible material.

19. The IV infiltration detection system of claim 14, wherein the visible border comprises a reflective or fluorescent coating.

20. The IV infiltration detection system of claim 14, wherein the visible border comprises a first color.

21. The IV infiltration detection system of claim 14, wherein the visible border comprises a third layer adhered to the first layer.

22. An Intravenous (IV) infiltration detection system, comprising:
a patch having a first side and a second side, with an adhesive disposed on a perimeter of the second side, the adhesive configured to secure the patch to skin of a patient around an IV insertion site; and
an absorbent material coupled to the first side of the patch, the absorbent material impregnated with a dye.

23. The IV infiltration detection system of claim 22, wherein the dye comprises methylene blue.

24. The IV infiltration detection system of claim 22, wherein the patch comprises a transparent center portion configured to enable view of an IV insertion site.

25. The IV infiltration detection system of claim 22, wherein the absorbent material comprises a moisture-wicking material that extends from a center portion of the patch to an edge of the patch.

26. The IV infiltration detection system of claim 25, wherein the absorbent material is impregnated with the dye at the edge of the patch.

27. The IV infiltration detection system of claim 22, wherein the absorbent material is disposed around a perimeter of the first side of the patch.

28. The IV infiltration detection system of claim 22, wherein the patch further comprises a first visible indicator, the IV infiltration detection system further comprising:
a second patch disposed on the second side of the patch, wherein:
the second patch comprises a second visible indicator;
in a first state the first visible indicator and second visible indicator produce a first pattern; and
in a second state the first visible indicator and second visible indicator produce a second pattern.

29. The IV infiltration detection system of claim 22, further comprising:
a first indicator strip formed of a flexible material, the first indicator strip comprising:
a first adhesive disposed on a first end of the first indicator strip; and
a visible indicator disposed on a second end of the first indicator strip; and
a second indicator strip formed of a flexible material, the second indicator strip comprising:
a second adhesive disposed on a first end of the second indicator strip, wherein
a second end of the second indicator strip defines a socket configured to slidably couple with the visible indicator of the first indicator strip and reveal the visible indicator in response to swelling of the skin of the patient.

30. The IV infiltration detection system of claim 22, wherein the second side of the patch comprises a visible border around an area of the patch and the patch further comprises:
a retainer adhered to the second side of the patch;
a layer slidably coupled with the retainer, wherein:
the layer comprises an opaque border configured to align with the visible border of the patch;
in a first state, the visible border is occluded by the opaque border; and
in a second state the visible border is at least partially visible around the opaque border.
